(19) United States
(12) Patent Application Publication
(10) Pub. No.: US 2020/0383704 A1
(43) Pub. Date: Dec. 10, 2020

(54) SPECIMEN RETRIEVAL DEVICES WITH FLEXIBLE ARMS FOR SUPPORTING COLLECTION BAGS

(71) Applicant: COVIDIEN LP, MANSFIELD, MA (US)

(72) Inventors: SAUMYA BANERJEE, HAMDEN, CT (US); JACOB C. BARIL, NORWALK, CT (US); GEORGE S. MATTA, PLAINVILLE, MA (US); MATTHEW A. DININO, NEWINGTON, CT (US); ROY J. PILLETERE, NORTH HAVEN, CT (US)

(21) Appl. No.: 16/430,604

(22) Filed: Jun. 4, 2019

(51) Int. Cl. A61B 17/50

(52) U.S. Cl. CPC A61B 17/50; A61B 2017/00867

(57) ABSTRACT

A specimen retrieval device includes an elongated shaft assembly and an end effector. The elongated shaft assembly has a proximal end portion and a distal end portion. The end effector is supported on the distal end portion of the elongated shaft assembly and includes a first arm and a second arm. The first arm defines a first arcuate profile having a first cross-section therealong. The second arm defines a second arcuate profile having a second cross-section therealong different than the first cross section. The first and second arms are configured to cooperate with one another to selectively support a collection bag thereon.

### TECHNICAL FIELD

This disclosure relates to surgical instruments, and more particularly, to specimen retrieval devices that support tissue collection bags.

### BACKGROUND

Specimen retrieval devices are commonly used during surgical procedures to collect and remove tissue specimens from a patient. Typically, during a surgical procedure in which tissue is transected, e.g., a hysterectomy procedure, a specimen retrieval device including a tissue collection bag is positioned to receive the tissue specimen once the tissue is transected. In some procedures, a grasper may be used to transfer the transected tissue specimen into the bag. Alternately, the bag may be positioned in relation to the tissue specimen to allow the tissue specimen to fall into the bag. Containment and extraction of large specimens in the gynecological space, for instance, can take between 5 minutes and 45 minutes, the length of which often depends on the size of the specimen. For example, large bulky specimens take longer to contain and extract because they are typically difficult to manipulate within a tight anatomical space.

### SUMMARY

The disclosure generally relates to specimen retrieval devices having end effectors with first and second arms that have different cross-sections for cooperating with one another to support a collection bag.

In aspects of the disclosure, a specimen retrieval device includes an elongated shaft assembly and an end effector. The elongated shaft assembly has a proximal end portion and a distal end portion. The end effector is supported on the distal end portion of the elongated shaft assembly and includes a first arm and a second arm. The first arm defines a first arcuate profile having a first cross-section therealong. The second arm defines a second arcuate profile having a second cross-section therealong different than the first cross section. The first and second arms are configured to cooperate with one another to selectively support a collection bag thereon.

In embodiments, the elongated shaft assembly may include an inner shaft that supports the end effector. The elongated shaft assembly may include an outer shaft. The inner shaft may be supported within the outer shaft. The inner shaft may be axially movable relative to the outer shaft. Axial movement of the inner shaft relative to the outer shaft may cause relative movement between the first and second arms. One or both of the first or second arms may include a flexible material. The flexible material may be a shape memory material. The shape memory material may include a nickel titanium alloy.

In some embodiments, proximal movement of the inner shaft relative to the outer shaft may cause the first and second arms to radially compress and retract into the outer shaft when the first and second arms are disposed distally of the outer shaft.

According to one aspect of this disclosure, a specimen retrieval device includes an elongated shaft assembly having a proximal end portion and a distal end portion. The specimen retrieval device further includes an end effector supported on the distal end portion of the elongated shaft assembly. The end effector includes a first arm and a second arm. The first arm defines a first arcuate profile having a first cross-section therealong. The second arm defines a second arcuate profile having a second cross-section therealong. The first cross-section is bigger than the second cross-section. The specimen retrieval device further includes a collection bag supported on the end effector and deployable through the elongated shaft assembly.

According to another aspect of this disclosure, a specimen retrieval device includes an elongated shaft assembly and an end effector. The elongated shaft assembly has a proximal end portion and a distal end portion. The end effector is supported on the distal end portion of the elongated shaft assembly and includes a first arm and a second arm. The first arm has a first cross-section. The second arm has a second cross-section that is smaller than the first cross-section to enable the first and second arms to move between a retracted position and a deployed position. The first and second arms are receivable within the elongated shaft assembly when in the retracted position, and when in the deployed position, the first and second arms are disposed in an annular arrangement to position a collection bag for receiving a specimen therein.

The details of one or more aspects of this disclosure are set forth in the accompanying drawings and the description below. Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective view of a specimen retrieval device in accordance with the principles of this disclosure, the specimen retrieval device illustrated in a retracted position;

FIG. 2 is a perspective view of the specimen retrieval device of FIG. 1 in a deployed position;

FIG. 3 is an enlarged, top view of an end effector of the specimen retrieval device of FIGS. 1 and 2;

FIG. 3A is a cross-sectional view of a portion of a first arm of the end effector of FIG. 3 as taken along section line 3A-3A of FIG. 3;

FIG. 3B is a cross-sectional view of a portion of a second arm of the end effector of FIG. 3 as taken along section line 3B-3B of FIG. 3; and

FIG. 4 is a side view of a distal end portion of the specimen retrieval device of FIGS. 1 and 2 with the specimen retrieval device illustrated in the deployed position of FIG. 2.

### DETAILED DESCRIPTION

Embodiments of the disclosed specimen retrieval devices are described in detail with reference to the drawings, in which like reference numerals designate identical or corresponding elements in each of the several views. As commonly known, the term “clinician” refers to a doctor (e.g., a surgeon), a nurse, or any other care provider and may include support personnel. Additionally, the term “proximal” refers to the portion of structure that is closer to the clinician and the term “distal” refers to the portion of structure that is farther from the clinician. In the following description, well-known functions or constructions are not described in detail to avoid obscuring this disclosure in unnecessary detail.

In general, this disclosure describes a specimen retrieval device having an end effector with flexible, retractable arms positioned to move relative to one another to facilitate quick and easy removal of collection bags supported on the arms. Advantageously, the retractable arms limit steps required for collection bag detachment/disengagement post specimen loading. For instance, the disclosed specimen retrieval device can reduce the complexity of large specimen loading (e.g., between about 500 g to 1500 g, or greater) while providing rigidity and maneuverability required to reduce specimen containment time. The specimen retrieval device can include an outer shaft (e.g., about 20-25 mm in diameter) that can receive a collection bag therethrough for deployment through a natural or artificial opening, such as a port or incision, to access an abdominal and/or vaginal cavity, for instance.

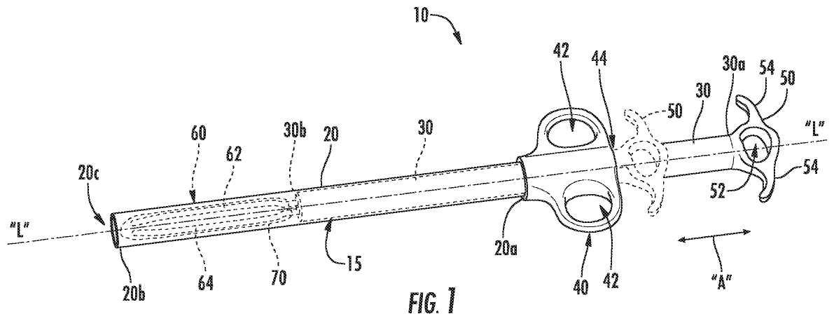

With reference to FIGS. 1-4, a specimen retrieval device 10 defines a longitudinal axis “L” and includes an elongated shaft assembly 15 having an outer shaft 20 and an inner shaft 30 supported within outer shaft 20. Outer shaft 20 has a proximal end portion 20a that supports a stationary handle 40 and a distal end portion 20b that defines a distal opening 20c. Stationary handle 40 defines finger openings 42 therethrough to facilitate grasping of stationary handle 40 by a user's fingers. Stationary handle 40 further defines a central opening 44 axially therethrough that is configured to slidably receive inner shaft 30 therein. Inner shaft 30 has a proximal end portion 30a that supports a movable handle 50 and a distal end portion 30b that supports an end effector 60. Movable handle 50 defines a finger opening 52 therethrough and includes wings 54 that extend from opposite sides of movable handle 50. Finger opening 52 and wings 54 are configured to receive a user's fingers to facilitate finger gripping. Movable handle 50 is positioned to move axially along longitudinal axis “L,” and relative to stationary handle 40, between distal and proximal positions, as indicated by arrows “A.”

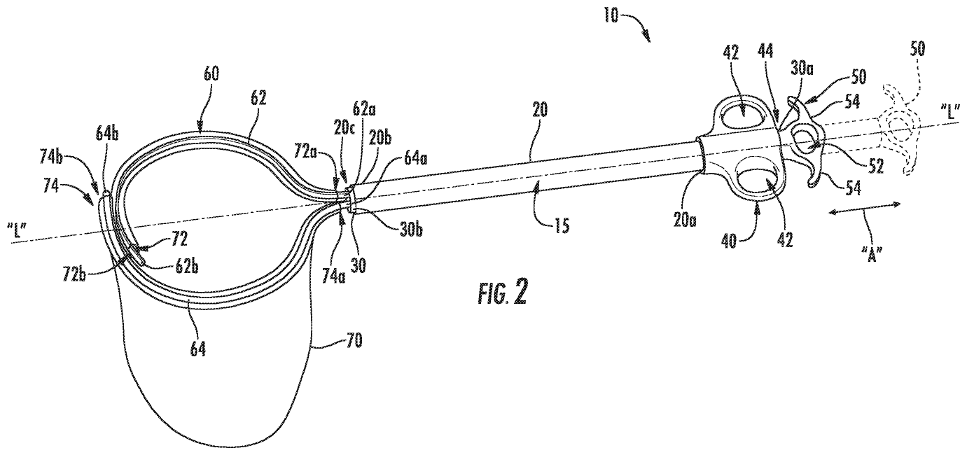

End effector 60 of specimen retrieval device 10 supports a collection bag 70 and includes a first arm 62 and a second arm 64 that at least partially overlap (e.g., in a longitudinal direction) and define a ring when deployed from outer shaft 20 (see FIG. 2) to provide increased leverage against applied force when loading a specimen into collection bag 70. Although the ring may have any suitable diameter, in some embodiments, the defined ring may have a diameter of about 5 inches to about 5.5 inches. First arm 62, which may have an arcuate profile such as a hook or question-mark shape, includes a proximal end portion 62a coupled to inner shaft 30 that curves distally to a distal end portion 62b that is free. Second arm 64, which may also have an arcuate profile such as hook or question mark shape, includes a proximal end portion 64a coupled to inner shaft 30 that curves distally to a distal end portion 64b that is free. First and second arms 62, 64 curve in opposite directions so as to be mirrored relative to one another about longitudinal axis “L.” As seen in FIGS. 3, 3A and 3B, first and second arms 62, 64 have different cross-sections. For instance, first arm 62 may have a thinner and/or smaller cross-section than second arm 64, or, in some embodiments, second arm 64 may have a thinner and/or smaller cross-section than first arm 62. This difference in cross-sections enables first and second arms 62, 64 to easily slide or cam along one another while providing overall rigidity needed to load large and/or heavy specimens into collection bag 70 while collection bag 70 is supported on end effector 60. Such cross-sectional differences may be along portions and/or entireties of first and/or second arms 62, 64.

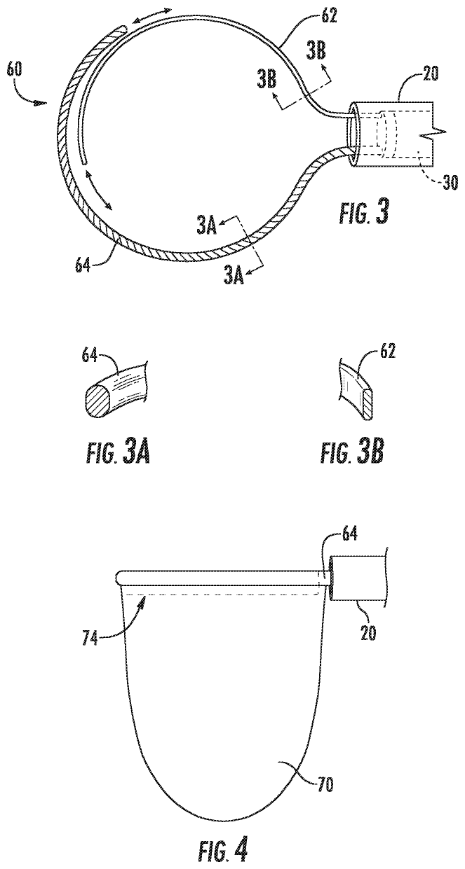

As seen in FIGS. 2 and 4, collection bag 70, which may include any suitable rollable material such as nylon, polyurethane, etc., may be selectively attached and/or removed from end effector 60. Collection bag 70 can be supported on end effector 60 and furled (e.g., tightly) around first and second arms 62, 64. Collection bag 70 includes a first arm channel 72 that has open proximal and distal ends 72a, 72b and second arm channel 74 with open proximal and distal ends 74a, 74b. First and/or second arm channels 72, 74 may be provided on collection bag 70 by any suitable technique such as suturing and/or welding. First arm channel 72 slidably receives first arm 62 of end effector 60 and second arm channel 74 slidably receives second arm 64 of end effector 60. First and second arm channels 72, 74 are configured to maintain first and second arms 62, 64 of end effector 60 in a closed loop. While collection bag 70 constrains first and second arms 62, 64 from overly flexing away from longitudinal axis “L,” first and second arms 62, 64 are configured to provide an outward force onto inner surfaces of collection bag 70 that define channels 72, 74 for maximizing bag opening when collection bag 70 is deployed from outer shaft 20.

As illustrated in FIGS. 1-4, specimen retrieval device 10 can be positioned for specimen retrieval, including, for example, insertion into a patient through a natural opening (e.g., vagina) or an artificial opening (e.g., incision, access portal, etc.). In order to facilitate insertion through narrow openings, end effector 60 is retained in a retraction position within outer shaft 20 of specimen retrieval device 10 with collection bag 70 supported on first and second arms 62, 64 of end effector 60 tightly furled about end effector 60, as seen in FIG. 1. Once inserted, end effector 60 and collection bag 70 can be deployed from outer shaft 20 by axially moving movable handle 50 of specimen retrieval device 10 toward stationary handle 40. Axial movement of movable handle 50 toward stationary handle 40 causes end effector 60 and collection bag 70 to pass through distal opening 20c so that first and second arms 62, 64 can move radially away from one another and the longitudinal axis “L” from a retracted position thereof (FIG. 1) to a deployed position (FIG. 2) where first and second arms 62, 64 define a closed loop or ring. First and second arms 62, 64 may be biased to move toward the deployed position, for instance, by virtue of shape memory material of first and/or second arms 62, 64, which may include a nickel titanium alloy, for example. As first and second arms 62, 64 move toward the deployed positions, first and second arms 62, 64 open collection bag 70. Collection bag 70 may restrain first and second arms 62, 64 and prevent or limit radially movement of first and second arms 62, 64 away from one another, for instance, beyond the deployed position.

Once one or more specimens are loaded into collection bag 70, specimen retrieval device 10 can be retracted in a single motion such that first and second arms 62, 64 can slide or cam over one another so that first and second arms 62, 64 move toward one another (e.g., radially) to facilitate retraction through a port, an incision, and/or other opening. In particular, the surfaces of the port or tissue defining the opening may act on first and second arms 62, 64 to compress first and second arms 62, 64 together in a radially inward direction toward longitudinal axis “L” as specimen retrieval device 10 is drawn through such opening. Alternatively, and/or additionally, movable handle 50 can be separated from stationary handle 40 so that first and second arms 62, 64 are drawn back (e.g., axially and radially) at least partially into outer shaft 20 to facilitate radial movement of first and second arms 62, 64 toward one another and retraction through a narrow opening.

With portions of collection bag 70 and any specimens contain therein still within the patient, but specimen retrieval device 10 otherwise removed from the patient, the clinician can then proceed to extract the remainder of collection bag 70 through the opening and being steps for specimen exteriorization and morcellation.

Securement of any of the components of the disclosed devices may be effectuated using known securement techniques such welding, crimping, gluing, heat-shrinking, fastening, etc.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Pat. No. 8,828,023, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that this disclosure is not limited to the precise embodiments described, and that various other changes and modifications may be effected by one skilled in the art without departing from the scope or spirit of this disclosure. Additionally, the elements and features shown or described in connection with certain embodiments may be combined with the elements and features of certain other embodiments without departing from the scope of this disclosure, and that such modifications and variations are also included within the scope of this disclosure. Accordingly, the subject matter of this disclosure is not limited by what has been particularly shown and described.

What is claimed is:

1. A specimen retrieval device, comprising:
an elongated shaft assembly having a proximal end portion and a distal end portion; and
an end effector supported on the distal end portion of the elongated shaft assembly and including a first arm and a second arm, the first arm defining a first arcuate profile having a first cross-section therealong, the second arm defining a second arcuate profile having a second cross-section therealong different than the first cross section, the first and second arms configured to cooperate with one another to selectively support a collection bag thereon.

2. The specimen retrieval device of claim 1, wherein the elongated shaft assembly includes an inner shaft that supports the end effector.

3. The specimen retrieval device of claim 2, wherein elongated shaft assembly includes an outer shaft, the inner shaft supported within the outer shaft.

4. The specimen retrieval device of claim 3, wherein the inner shaft is axially movable relative to the outer shaft.

5. The specimen retrieval device of claim 4, wherein axial movement of the inner shaft relative to the outer shaft causes relative movement between the first and second arms.

6. The specimen retrieval device of claim 5, wherein at least one of the first or second arms includes a flexible material.

7. The specimen retrieval device of claim 6, wherein both the first and second arms include flexible material.

8. The specimen retrieval device of claim 6, wherein the flexible material is a shape memory material.

9. The specimen retrieval device of claim 8, wherein the shape memory material includes a nickel titanium alloy.

10. The specimen retrieval device of claim 5, wherein proximal movement of the inner shaft relative to the outer shaft causes the first and second arms to radially compress and retract into the outer shaft when the first and second arms are disposed distally of the outer shaft.

11. A specimen retrieval device, comprising:
an elongated shaft assembly having a proximal end portion and a distal end portion;
an end effector supported on the distal end portion of the elongated shaft assembly and including a first arm and a second arm, the first arm defining a first arcuate profile having a first cross-section therealong, the second arm defining a second arcuate profile having a second cross-section therealong, the first cross-section being bigger than the second cross-section; and
a collection bag supported on the end effector and deployable through the elongated shaft assembly.

12. The specimen retrieval device of claim 11, wherein the elongated shaft assembly includes an inner shaft that supports the end effector.

13. The specimen retrieval device of claim 12, wherein elongated shaft assembly includes an outer shaft, the inner shaft supported within the outer shaft.

14. The specimen retrieval device of claim 13, wherein the inner shaft is axially movable relative to the outer shaft.

15. The specimen retrieval device of claim 4, wherein axial movement of the inner shaft relative to the outer shaft causes relative movement between the first and second arms.

16. The specimen retrieval device of claim 15, wherein at least one of the first or second arms includes a flexible material.

17. The specimen retrieval device of claim 16, wherein the flexible material is a shape memory material.

18. The specimen retrieval device of claim 17, wherein the shape memory material includes a nickel titanium alloy.

19. The specimen retrieval device of claim 15, wherein proximal movement of the inner shaft relative to the outer shaft causes the first and second arms to radially compress and retract into the outer shaft when the first and second arms are disposed distally of the outer shaft.

20. A specimen retrieval device, comprising:
an elongated shaft assembly having a proximal end portion and a distal end portion; and
an end effector supported on the distal end portion of the elongated shaft assembly and including a first arm and a second arm, the first arm having a first cross-section, the second arm having a second cross-section that is smaller than the first cross-section to enable the first and second arms to move between a retracted position and a deployed position, the first and second arms receivable within the elongated shaft assembly when in the retracted position, and when in the deployed position, the first and second arms are disposed in an annular arrangement to position a collection bag for receiving a specimen therein.
